(19) United States
(12) Patent Application Publication
(10) Pub. No.: US 2011/0028707 A1
(43) Pub. Date: Feb. 3, 2011

(54) KETONE ACCUMULATION INHIBITOR

(75) Inventors: Masahiro Okuno, Kanagawa (JP); Koji Suzuki, Tokyo (JP)

Correspondence Address: GRIFFIN & SZIPL, PC, SUITE PH-1, 2300 NINTH STREET, SOUTH, ARLINGTON, VA, 22204, US

(73) Assignee: MITSUI SUGAR CO., LTD., Tokyo (JP)

(21) Appl. No.: 12/935,675

(22) PCT Filed: Mar. 25, 2009
(86) PCT No.: PCT/JP2009/055978

§ 371 Date: Sep. 30, 2010

(30) Foreign Application Priority Data
Mar. 31, 2008 (JP) P2008-093186

(51) Int. Cl. C07H 3/04

(52) U.S. Cl. 536/123

(57) ABSTRACT

An object of the present invention is to provide a ketone body accumulation inhibitor which inhibits the accumulation of ketone bodies caused by exercising. The present invention provides a ketone body accumulation inhibitor containing isomaltulose as an active ingredient.

### TECHNICAL FIELD

The present invention relates to a ketone body accumulation inhibitor and particularly a ketone body accumulation inhibitor which inhibits the accumulation of ketone bodies caused by exercising in diabetic patients, patients with metabolic syndrome, and obese persons.

### BACKGROUND ART

Ketosis represents the state in which ketone bodies are increased in the body, and it is caused by the fact that lipid metabolism, instead of carbohydrate metabolism, is promoted to produce excessive ketone bodies by diabetes, fasting, exercising and the like. Since ketone bodies are acidic, progression of the ketosis causes blood to shift to acid (ketoacidosis) and causes disordered consciousness or severe coma in the serious cases, and can cause death.

A mechanism of ketone body production is considered as follows. Fatty acids released from neutral fat turn into acyl-CoAs in the liver. The acyl-CoAs combine with glycerol to resynthesize neutral fat when insulin is sufficient, but on the other hand, they are β-oxidized into acetyl-CoA when the insulin is deficient. When the acetyl-CoA is excessive, or gluconeogenesis is promoted by the shortage of carbohydrates in the body by fasting, diabetes and the like, ketone bodies are produced from the acetyl-CoA (Non-Patent Documents 1 and 2). Ketone bodies tend to increase in persons who do not usually exercise, while they do not tend to increase in persons who usually work out (Non-Patent Document 2).

Administration of insulin or sugar such as glucose is generally conducted for prevention and treatment of ketosis. However, since diabetic patients fall into the state of high blood sugar levels when they take sugars such as glucose, sugar intake is restricted for them. Though diabetic patients need to improve their physical conditions by exercising, they may have difficulties in exercising without anxieties due to the risk of high blood sugar levels, ketosis and acidosis. Also, when patients with metabolic syndrome and obese persons take sugars such as glucose to prevent accumulation of ketone bodies caused by high-intensity exercising, oxidative metabolism of lipids by exercising is inhibited, and hence they may not get sufficient exercising effects.

On the other hand, fructose intake causes little elevation of blood sugar levels, but it is not suitable to supply muscles with energy because a large amount of fructose intake is metabolized to lipids in the liver (Non-Patent Document 3).

A ketone body urinary excretion accelerator containing diglyceride as an active ingredient is known as an eliminant for ketone derivative into urine (Patent Document 1).

Patent Document 1: Japanese Patent Application Laid-Open Publication No. 8-26988

Non-Patent Document 1: Examination and Technology Vol. 32, No. 3, pp 276-277, 2004

Non-Patent Document 2: Japanese Journal of Biomechanics in Sports and Exercise Vol. 1, No. 1, pp 105-119, 1997

Non-Patent Document 3: Nutrition Reviews Vol. 63, No. 5, pp 133-157, 2005

### DISCLOSURE OF THE INVENTION

### Technical Problem

In such a situation, for subjects such as diabetic patients, patients with metabolic syndrome, and obese persons who have an accumulation of ketone bodies caused by exercising, an agent to prevent accumulation of ketone bodies caused by exercising and to allow them to get sufficient exercising effects is required. Hence, the object of the present invention is to provide a ketone body accumulation inhibitor. Further, the present invention aims to provide an agent which inhibits accumulation of ketone bodies caused by exercising in diabetic patients, patients with metabolic syndrome, and obese persons.

### Solution to Problem

The present invention provides a ketone body accumulation inhibitor containing isomaltulose as an active ingredient. The ketone body accumulation inhibitor of the present invention is characterized by inhibiting accumulation of ketone bodies caused by exercising.

The ketone body accumulation inhibitor of the present invention can inhibit accumulation of ketone bodies caused by exercising in subjects such as diabetic patients, patients with metabolic syndrome, and obese persons who have an accumulation of ketone bodies caused by exercising. Moreover, it can inhibit accumulation of ketone bodies, maintaining oxidative metabolism of lipids caused by exercising to maintain sufficient exercising effects.

Furthermore, the present invention provides a method for inhibiting accumulation of ketone bodies caused by exercising, comprising a step of applying the ketone body accumulation inhibitor containing isomaltulose as an active ingredient to the subjects.

### BRIEF DESCRIPTION OF THE DRAWINGS

FIG. 1 is a graph showing blood glucose levels changes over time by drink intake after exercising;

FIG. 2 is a graph showing blood insulin levels changes over time by drink intake after exercising;

FIG. 3 is a graph showing blood C-peptide levels changes over time by drink intake after exercising;

FIG. 4 is a graph showing blood free fatty acid levels changes over time by drink intake after exercising;

FIG. 5 is a graph showing blood lactic acid levels changes over time by drink intake after exercising;

FIG. 6 is a graph showing respiratory quotient changes over time by drink intake after exercising;

FIG. 7 is a graph showing the amount of oxidized carbohydrate changes over time by drink intake after exercising;

FIG. 8 is a graph showing the amount of oxidized fat changes over time by drink intake after exercising;

FIG. 9 is a graph showing blood neutral fatty acid levels changes over time by drink intake after exercising;

FIG. 10 is a graph showing blood epinephrine levels changes over time by drink intake after exercising;

FIG. 11 is a graph showing blood nolepinephrine levels changes over time by drink intake after exercising;

FIG. 12 is a graph showing blood 3-OHBA levels changes over time by drink intake after exercising; and

FIG. 13 is a graph showing blood total ketone bodies levels changes over time by drink intake after exercising.

### BEST MODES FOR CARRYING OUT THE INVENTION

The preferred embodiments of the present invention will be described in detail below.

In the present invention, “isomaltulose” is also referred to as palatinose and it is a disaccharide formed by an α-1,6-glucosyl bond between glucose and fructose. “Palatinose” is a registered trademark by Mitsui Sugar Co., Ltd.

Palatinose may be in the form of hydrate. Palatinose monohydrate has melting point of 123° C. to 124° C., specific optical rotation of [α]²⁰_{D}=+97.0−99.0° (C.=0.04), Fehling solution reducing ability of 52% of glucose and solubility into 100 g water of 38.4 g at 20° C. Moreover, sweetness quality of its aqueous solution is good and its degree of sweetness is about 40% of sucrose.

Palatinose is found in honey in nature. It also exists among transfer products produced by the action of α-glucosyltransferase (isomaltulose synthase) derived from bacteria and yeast on sucrose.

In industrial production, palatinose is produced by effects of α-glucosyltransferase derived from bacteria such as Protaminobacter rubrum or Serratia plymuthica on sucrose.

The ketone body accumulation inhibitor of the present invention is a ketone body accumulation inhibitor containing palatinose as an active ingredient and it is used for preventing accumulation of ketone bodies in subjects who produce and accumulate ketone bodies in the body by exercising. The subjects include humans who accumulate ketone bodies by exercising, particularly, diabetic patients, patients with metabolic syndrome and obese persons, and subjects suspected thereof. Patients with metabolic syndrome are for example, subjects who fit “definition and diagnostic criteria of metabolic syndrome” announced at the 102nd meeting of the Japanese Society of Internal Medicine (held in April 2005). Obese persons are for example, subjects who have BMIs of 25.0 or more. The criteria described above are original Japanese criteria, but applying the ketone body accumulation inhibitor of the present invention is not limited to Japanese.

The term “ketone body” is a generic term for acetone, acetoacetic acid and 3-hydroxybutyric acid (3-OHBA). Acetoacetic acid is synthesized from acetyl-CoA as the material. Acetoacetic acid is reduced to 3-hydroxybutyric acid, followed by decarboxylation to produce acetone. Acetoacetic acid and 3-hydroxybutyric acid are present in blood. When the use of sugar is insufficient (severe diabetes, starvation, a shortage of sugar intake and the like), blood ketone body levels increase, and a part of the ketone bodies are excreted in expiration or urine. The amounts of produced and accumulated ketone bodies in the body can be examined by measuring blood ketone bodies, ketone bodies in urine and/or acetone in expiration. As these measuring methods, methods known in the art can be used. Ketone bodies in the body are normally negative but they may turn to be positive by high-intensity exercising. Also, while in case of ketone bodies being detected at ordinary times in diabetic patients, they may be under poorly controlled conditions.

The ketone body accumulation inhibitor of the present invention is required to comprise palatinose as an active ingredient and it may be consisted of palatinose only or mixtures of palatinose and other ingredients. Other ingredients include known pharmaceutically acceptable excipients and carriers and in addition, they may include sucrose, flour, starch, dextrine, high-fructose corn syrup (HFCS) and the like. In the mixtures described above, the weight ratio of palatinose is preferably 99.99% to 10%, and more preferably, 99.99% to 20%.

As the ketone body accumulation inhibitor described above, a mixture of palatinose and other carbohydrates which are nonreducing sugars may be used. This reduces causes of coloration so that food may be less colored. Further, platinose may be combined with other carbohydrates with high solubility. This leads to less crystallization.

Palatinose may be used in the combination with sweeteners such as sucrose and high-fructose corn syrup. This decreases sweetness of sucrose, glucose and high-fructose corn syrup to produce food products with light and low sweetness.

As the ketone body accumulation inhibitor of the present invention, for instance, crystalline palatinose, powdered palatinose, palatinose syrup and/or trehalulose syrup can be used. Crystalline palatinose (Crystalline Palatinose-IC, trade name, Mitsui Sugar Co., Ltd.) and powdered palatinose (Palatinose Powder-ICP, trade name, Mitsui Sugar Co., Ltd.) include 99.0% or more of palatinose (including crystalline water). Palatinose syrups (Palatinose Syrup-ISN and Palatinose Syrup-TN, trade name, Mitsui Sugar Co., Ltd.) include 11% to 17% of palatinose and 53% to 59% of trehalulose. Trehalulose syrup (Mildear-75 and Mildear-85, trade name, Mitsui Sugar Co., Ltd.) includes 8% to 13% of palatinose and 83% to 89% of trehalulose.

The form of the ketone body accumulation inhibitor described above is not particularly limited as long as it contains palatinose as an active ingredient, and it includes a fondant, granule, tablet, syrup, ampuled liquid medicine, refreshing drink, jelly drink, powder drink and the like. Moreover, the ketone body accumulation inhibitor of the present invention can be combined with materials which are usable for food, quasi-drug and medicine, and processed to food for specialized health uses, functional food, health food, quasi drug, medicine and the like for use.

The ketone body accumulation inhibitor of the present invention is preferably took after exercising. In taking palatinose, a fondant, granule, tablet, syrup or the like containing palatinose as an active ingredient may be took as it is, and it is preferably took with water, or dissolved or suspended in water to be taken as an ampuled liquid medicine or refreshing drink. The quantity of water to dissolve palatinose can be properly decided and 25 ml to 1000 ml of water is preferably used per 10 g of a mixture containing palatinose as an active ingredient and more preferably, 40 ml to 500 ml is used. Palatinose intake is preferably 0.5 g or more per kg of body weight, more preferably 0.7 g or more, and still more preferably 1 g or more. The intake method of the ketone body accumulation inhibitor of the present invention includes oral intake.

### EXAMPLES

The preferred examples of the present invention will be described in detail below but the present invention is not limited to these examples.

### Example 1

### Drinking Test

To male subjects with suspected metabolic syndrome and 85 cm or more of abdominal circumferences who lack in exercise, the following test was conducted. Table 1 shows the physical measurements and metabolic profiles of the subjects. The subjects, who had family histories of cardiopathy or type 2 diabetes and/or had dyslipidemia, were eliminated from the test. Palatinose or high-fructose corn syrup was dissolved in 500 ml of water so that its intake per kg of body weight could be 1.0 g of the solid, and sucralose, a high intensity sweetener, was dissolved in 500 ml of water so that its intake per kg of body weight could be 1.33 mg of the solid that gives the degree of sweetness between the high-fructose corn syrup and palatinose solutions, to prepare three kinds of test drinks. For these three kinds of drinks, drinking tests were conducted, respectively and each test was conducted at intervals of ten days or longer. The high-fructose corn syrup is composed of glucose and fructose, the same constituent sugars as sucrose and palatinose, and its calorie per the solid is 4 kcal/g which is the same value as that of sucrose and palatinose.

TABLE 1

| Profile | Value | |
|---|---|---|
| Age (years old) | 46.0 ± 2.3 | (38-59) |
| Body Height (cm) | 171.2 ± 1.9 | (161.1-182.5) |
| Body Weight (kg) | 79.4 ± 2.8 | (72.1-100.2) |
| BMI (kg/m²) | 27.1 ± 1.0 | (23.1-32.7) |
| Abdominal Circumference (cm) | 95.9 ± 1.4 | (91.8-102.6) |
| Body Fat (%) | 25.7 ± 1.2 | (22.8-30.6) |
| VO₂max (ml/kg/min) | 32.0 ± 1.2 | (25.7-35.9) |
| 50% VO₂ max (ml/kg/min) | 16.0 ± 0.6 | (12.9-18.0) |
| Exercise Tolerance VO₂ max (kp) | 2.95 ± 0.2 | (2.0-3.5) |
| Exercise Tolerance 50% VO₂ max (kp) | 1.1 ± 0.1 | (1.0-1.25) |
| Exercise Tolerance VO₂ max (watts) | 177.0 ± 9.9 | (120-210) |
| Exercise Tolerance 50% VO₂ max (watts) | 66.0 ± 3.5 | (60-75) |
| Fasting Blood Sugar (mg/dl) | 100.2 ± 4.8 | (81-132) |
| Fasting Insulin (μU/ml) | 9.8 ± 2.7 | (3.9-32.0) |
| HOMA-R | 2.5 ± 0.8 | (0.95-8.35) |
| Glycosylated Hemoglobin (%) | 5.4 ± 0.2 | (4.8-6.5) |
| Total Cholesterol (mg/dl) | 207 ± 10.8 | (146-254) |
| Neutral Fat (mg/dl) | 152 ± 11.0 | (94-212) |
| HDL Cholesterol (mg/dl) | 48.2 ± 1.7 | (42.9-59.0) |
| LDL Cholesterol (mg/dl) | 134 ± 6.7 | (111-166) |
| AST (IU/L) | 29.8 ± 4.1 | (17-52) |
| ALT (IU/L) | 44.5 ± 9.2 | (16-102) |
| γ-GTP (IU/L) | 54.4 ± 11.8 | (20-118) |
| Systolic Blood Pressure (mmHg) | 116 ± 5.1 | (94-142) |
| Diastolic Blood Pressure (mmHg) | 77 ± 4.1 | (60-96) |

Values are shown as the means ± SEM of 10 subjects.

Here, ALT, AST and γ-GTP are aspartate aminotransferase, alanine aminotransferase and γ-glutamyl transpeptidase, respectively. HOMA-R (Homeostasis Model Assessment insulin resistance index) is an index representing insulin resistance and represented by the following formula:

HOMA-R=Fasting Blood Sugar (mg/dl)×Fasting Blood Insulin Level (μU/ml)÷405

The method of physical measurement for the subjects was based on ACSM guideline (American College of Sports Medicine, 2001). Respiratory gas was collected and analyzed by a respiratory gas automatic analyzer (Oxycon Alpha, manufactured by Mijnhardt) every 30 seconds during the test. Maximal oxygen uptake (VO₂max) was calculated by increasing exercise loading gradually with a bicycle ergometer (818E, manufactured by Monark).

The tests were conducted as a double-blind crossover tests and dietary restriction was imposed on the subjects from two days before the tests. All subjects were instructed to abstain from alcohol, caffeine, green tea and high-intensity exercising 24 hours before the tests. Moreover, they recorded food intake two days before the first tests and repeated the same food intake before the second tests. This is for standardizing of the food intake before the tests and for minimizing the change of glycogen storage. All subjects took the same food as the dinner a day before all tests (725 kcal, 60% of the energy from carbohydrate, 30% from lipid and 10% from protein). After an overnight (10 hours to 12 hours) fast, the tests were conducted. During the fasting period, they were permitted to take water freely.

In order to avoid the effect of circadian rhythm, the tests were started at the same time. After the 15 minutes rest, the first venous blood sampling was conducted. The subjects exercised for an hour with loading of about 50% of VO₂ max by a bicycle ergometer. After exercising, blood sampling was conducted and they took the test drinks for 5 minutes. Blood sampling was conducted at time points of 10, 20, 30, 45, 60, 75, 90, 105 and 120 minutes after the test drink was taken in. The collected blood samples were submitted for various biochemical measurements. After the drink intake, their heart rates were continuously measured with an electrocardiograph (Dyan Scope, manufactured by Fukuda Denshi).

On the assumption that oxidation of proteins during exercising can be ignored, oxidation of total carbohydrates and lipids was calculated from VO₂ and VCO₂ using Frayn's equation. The VO₂ and VCO₂ represent oxygen intake and carbon dioxide excretion in L/min, respectively. Respiratory quotient (RQ) was calculated from oxygen intake and carbon dioxide excretion. Calorie consumption rate was calculated from RQ and VO₂ using Lusk's equation.

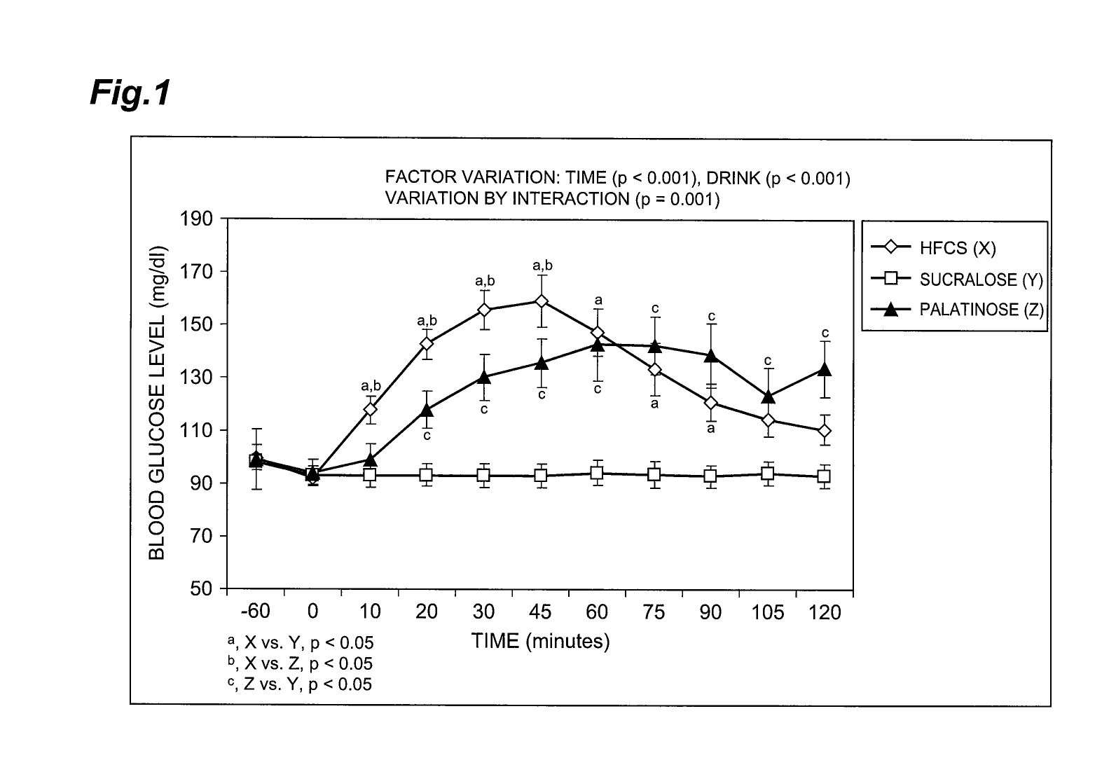

FIG. 1 shows blood glucose levels changes over time by drink intake after exercising. As shown in FIG. 1, in taking sucralose, a blood sugar level did not rise since no sugar was taken. In taking high-fructose corn syrup, a blood sugar level rose immediately thereafter and started to drop 45 minutes thereafter. In taking palatinose, a blood sugar level rose slowly thereafter, surpassed that in the case of taking high-fructose corn syrup around 60 minutes thereafter, and continued maintaining a relatively high value.

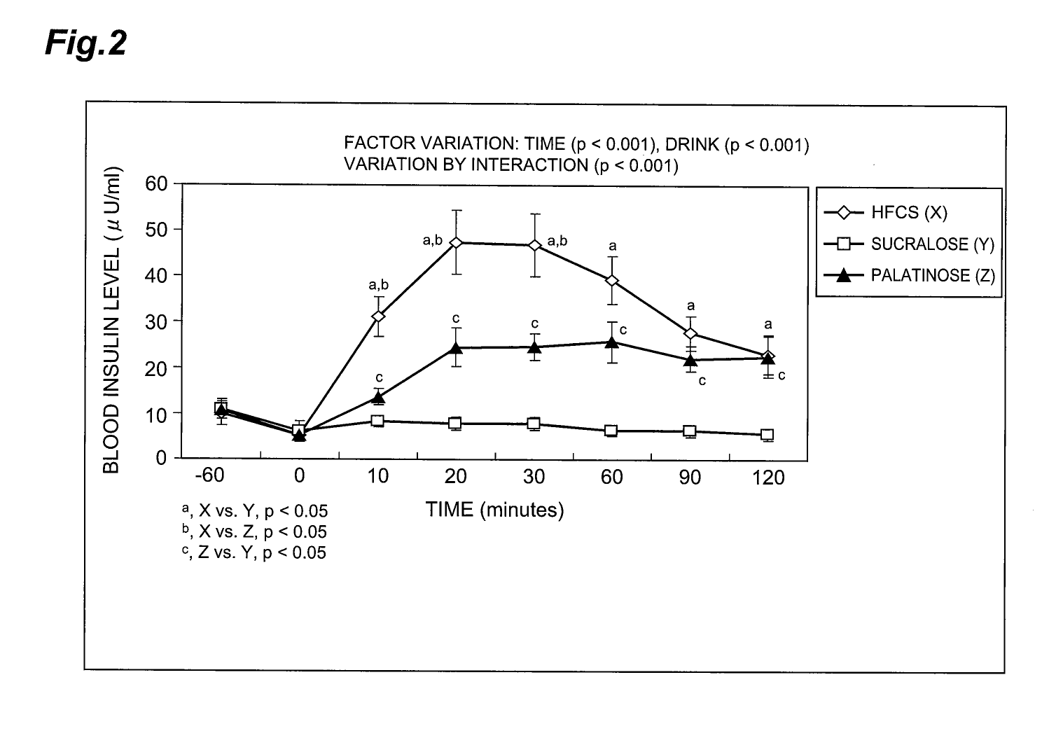

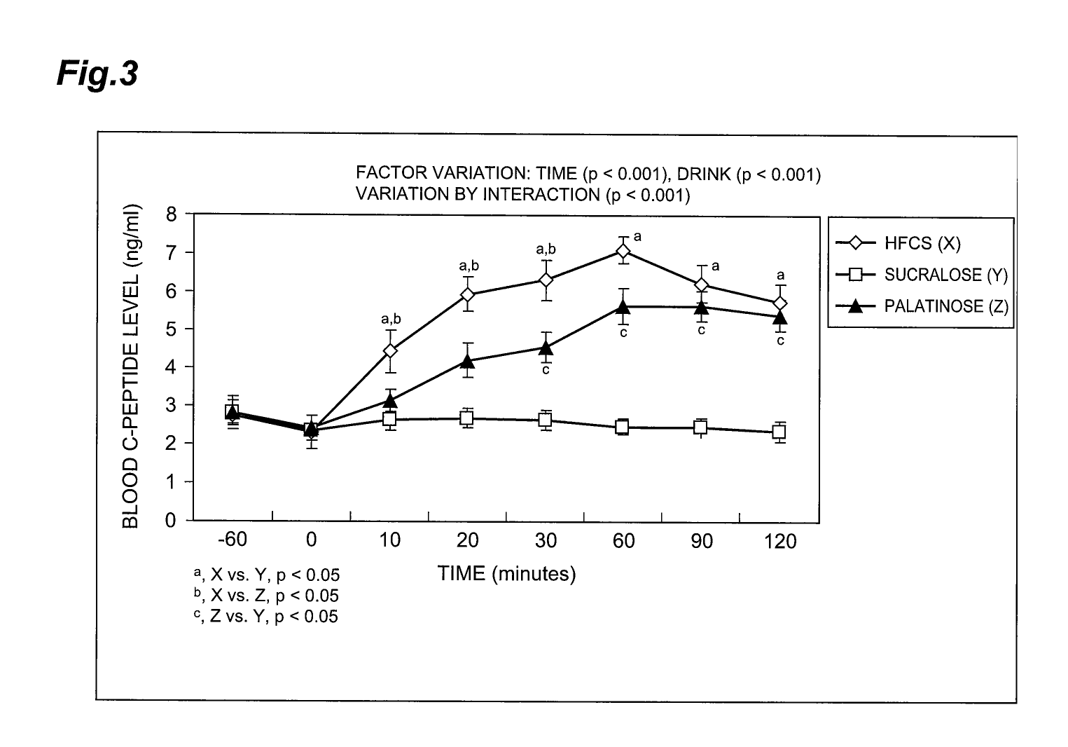

FIG. 2 shows blood insulin levels changes over time by drink intake after exercising. It was confirmed that insulin secretion was mild in taking palatinose while it was stimulated in taking high-fructose corn syrup. FIG. 3 shows blood C-peptide levels changes over time by drink intake after exercising. Since C-peptide is an index of insulin secretion, FIG. 3 shows a tendency similar to FIG. 2.

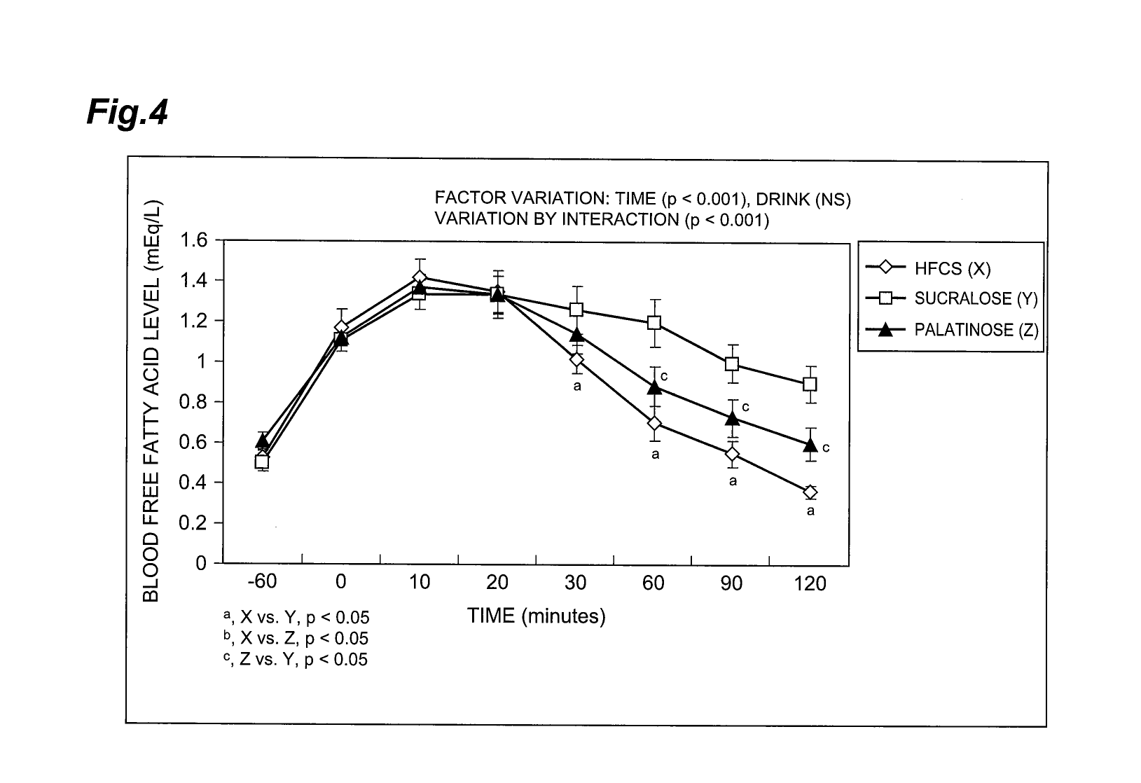

FIG. 4 shows blood free fatty acid levels changes over time by drink intake after exercising. Though free fatty acid levels rose after exercising as shown in FIG. 4 due to promoted metabolism of lipid by exercising, in taking high-fructose corn syrup, a free fatty acid level dropped 30 minutes thereafter. This is thought to result from a switch to metabolism of taken sugar. In taking sucralose, the higher values were maintained. In taking palatinose, the values were shown between those in taking high-fructose corn syrup and in taking sucralose.

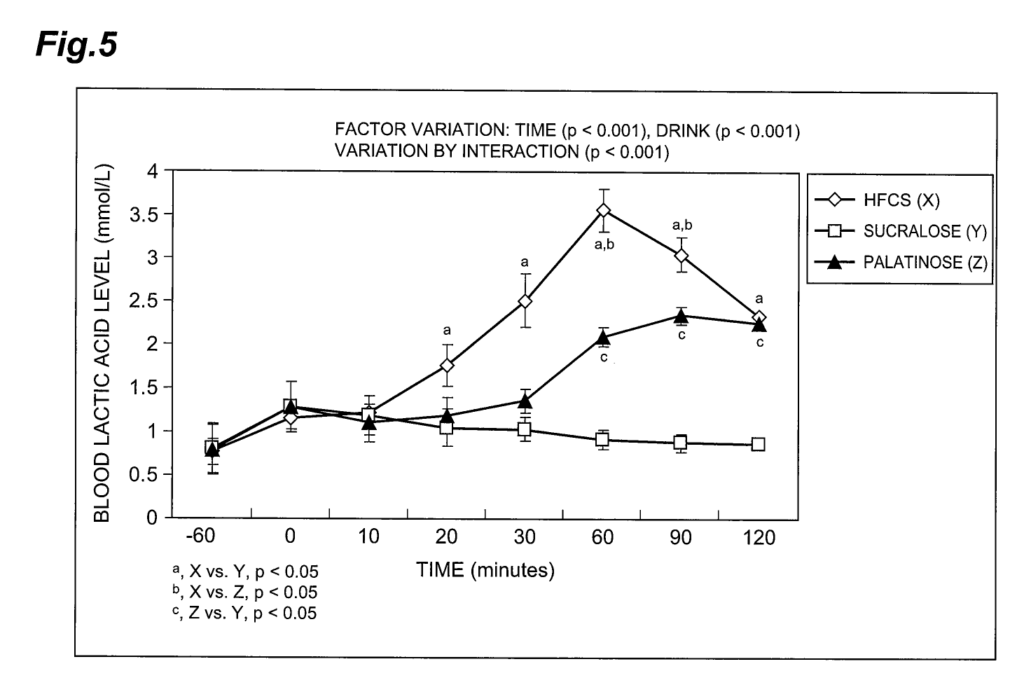

FIG. 5 shows blood lactic acid levels changes over time by drink intake after exercising. It was found that high-fructose corn syrup entered in glycolytic metabolism after its intake since a lactic acid level rose in taking it. On the other hand, in taking sucralose, there was no change. In taking palatinose, the values were shown between those in taking high-fructose corn syrup and in taking sucralose.

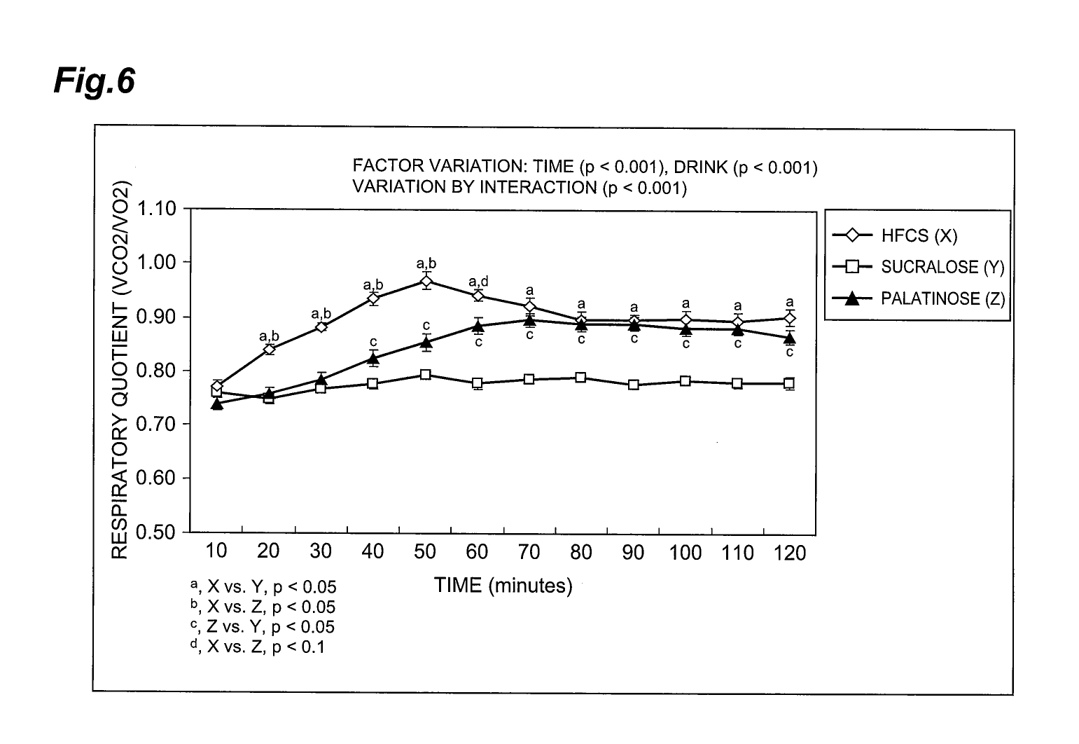

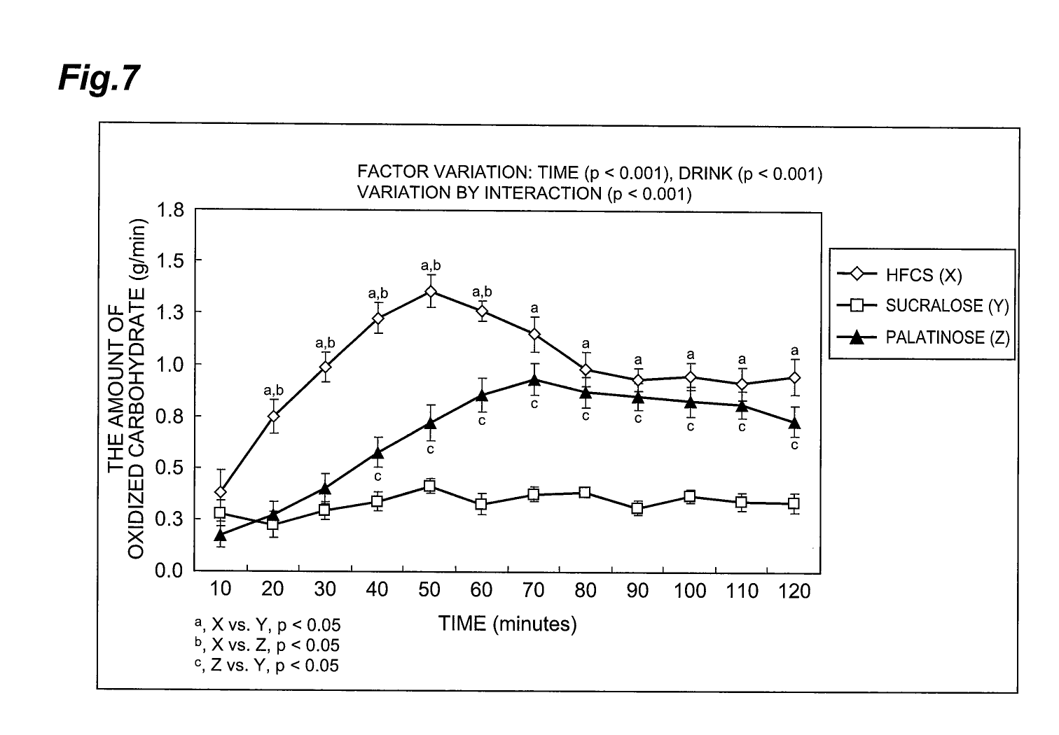

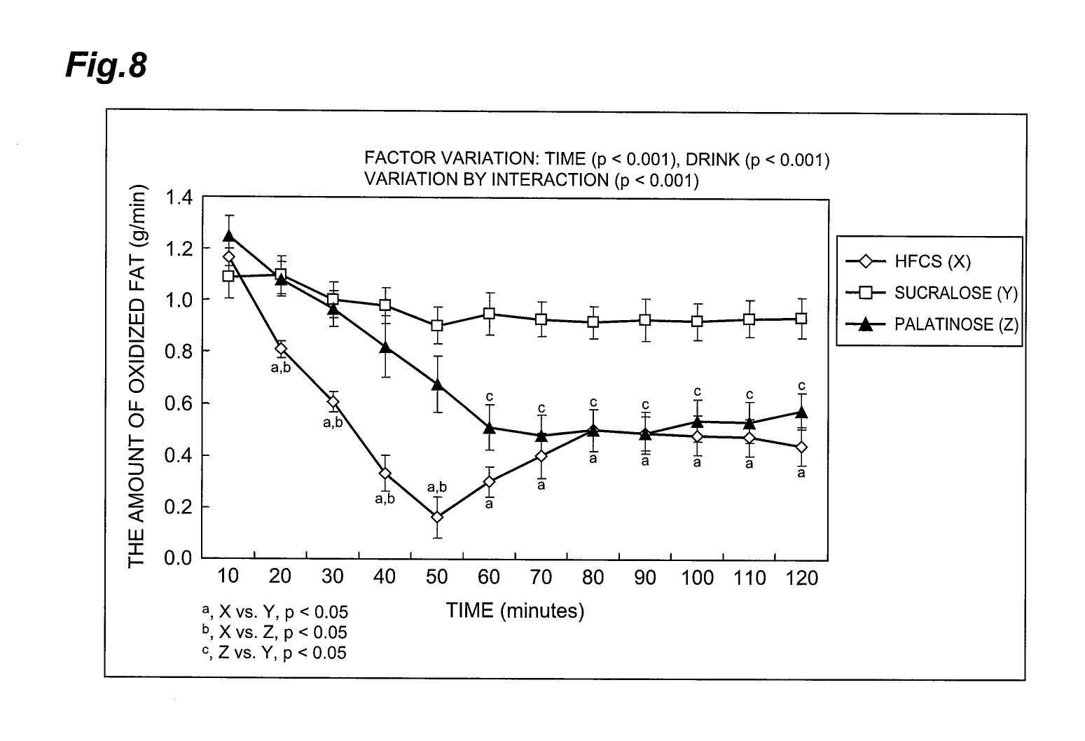

The changes over time of respiratory quotient, the amount of oxidized carbohydrate and the amount of oxidized lipids provided by drink intake after exercising are shown in FIGS. 6, 7 and 8, respectively. The respiratory quotient approximates to 1.0 when carbohydrates are oxidized, while the respiratory quotient approximates to 0.7 when lipids are oxidized. As shown in FIG. 6, in taking sucralose, the respiratory quotient close to 0.7 was maintained which shows that lipids were metabolized. In taking high-fructose corn syrup, it was found that the metabolism of lipids was replaced by the metabolism of carbohydrates since the respiratory quotient rose to the value close to 1.0. In taking palatinose, the values were shown between those in taking high-fructose corn syrup and in taking sucralose. As shown in FIG. 7, the amount of oxidized carbohydrate showed higher values in taking high-fructose corn syrup, while showed lower values in taking sucralose. In taking palatinose, the values were shown between those of high-fructose corn syrup and sucralose. The amount of oxidized lipids in FIG. 8 showed lower values in taking high-fructose corn syrup while higher values in taking sucralose, contrary to the results for the amount of oxidized carbohydrate. In taking palatinose, the values were shown between those of high-fructose corn syrup and sucralose. It is recognized from FIGS. 4, 5, 7 and 8 that lipid oxidation is rather difficult to prevent in taking palatinose while lipid oxidation is maintained in taking sucralose and it is replaced by carbohydrate oxidation in taking high-fructose corn syrup.

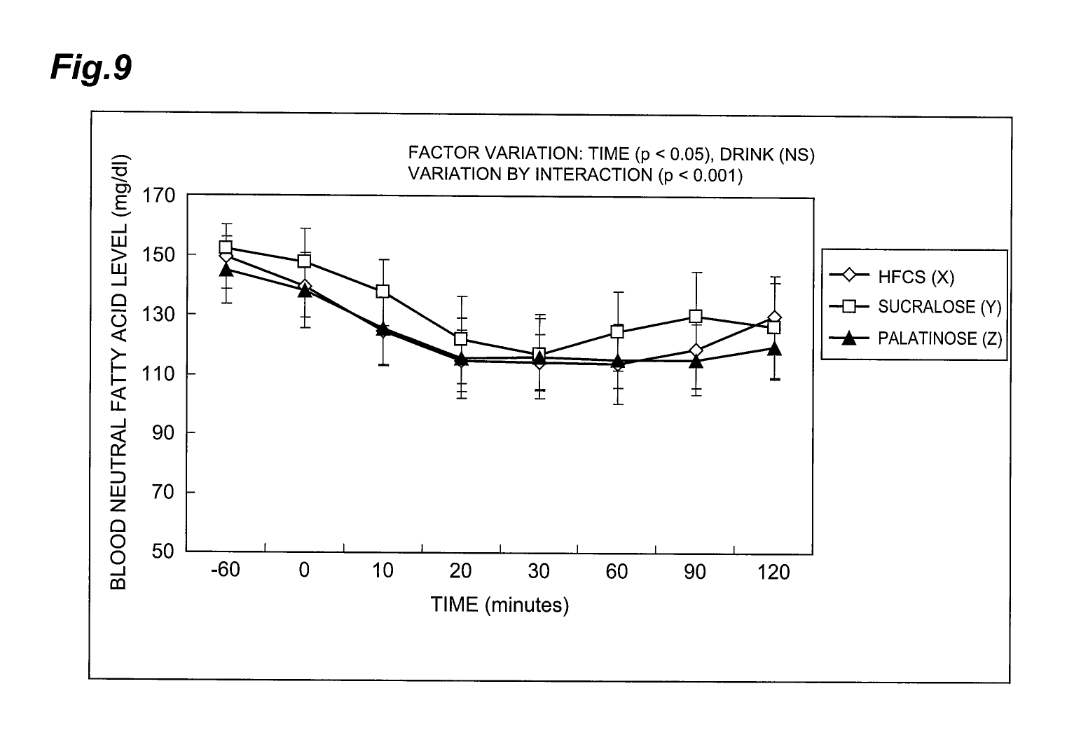

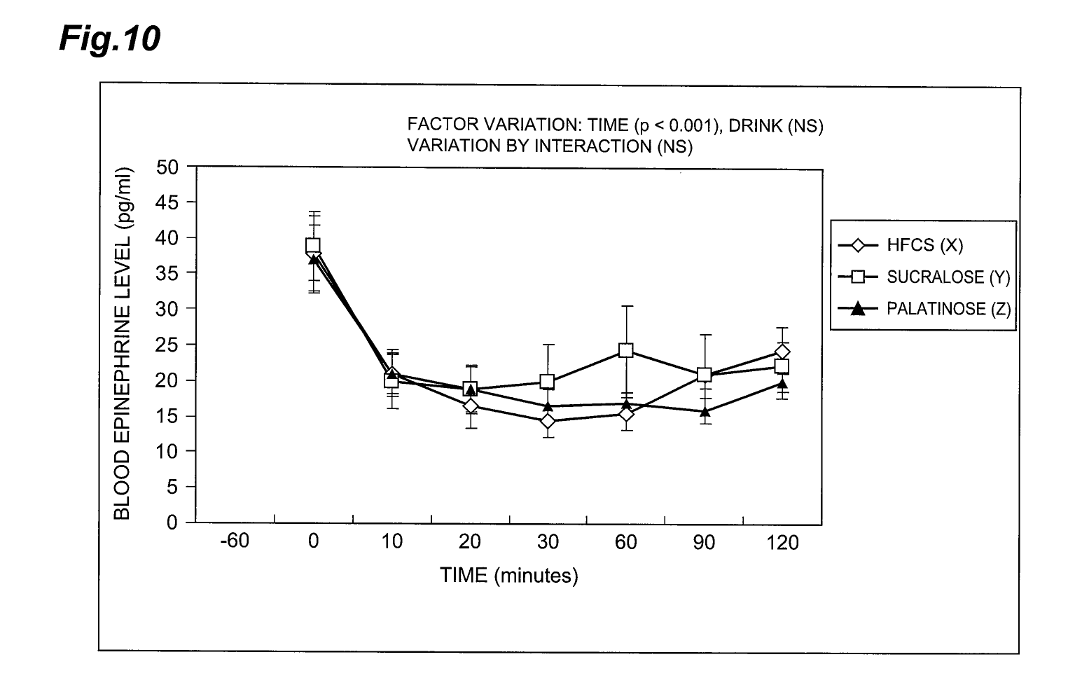

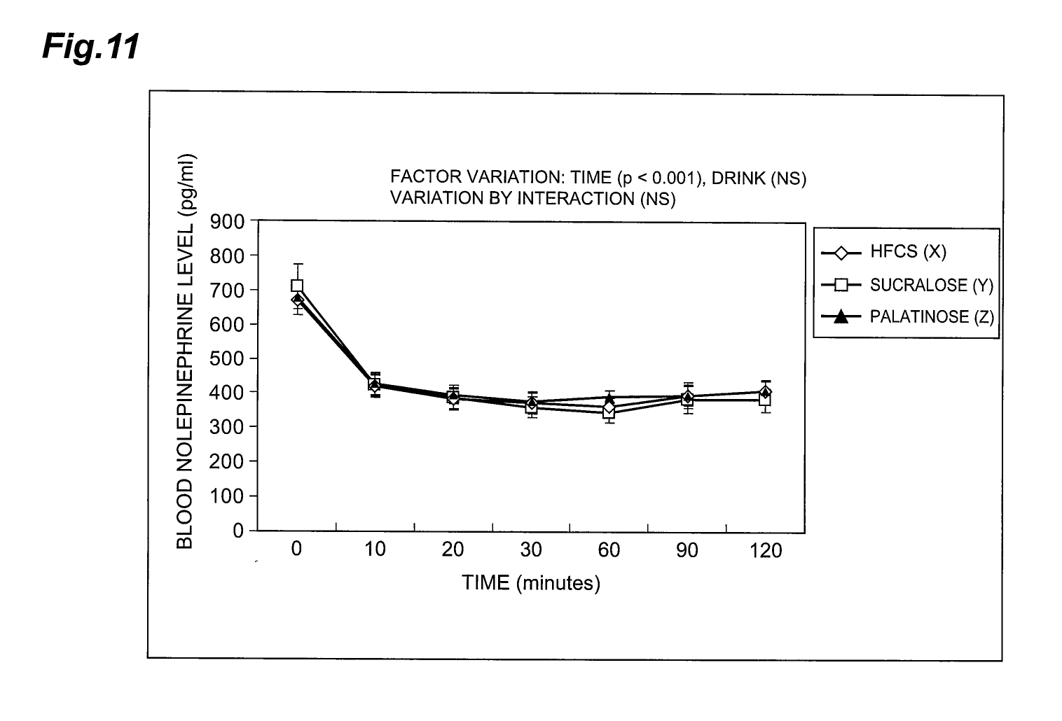

FIG. 9 to FIG. 11 show changes over time of blood neutral fat levels, blood epinephrine levels and blood nolepinephrine levels by drink intake after exercising. Epinephrine and nolepinephrine have an effect of promoting fat metabolism. In neutral fat, epinephrine and nolepinephrine, there were no differences between each drink. It was found that taking high-fructose corn syrup or palatinose had no effect on the metabolic pathway in which neutral fat, epinephrine and nolepinephrine participated.

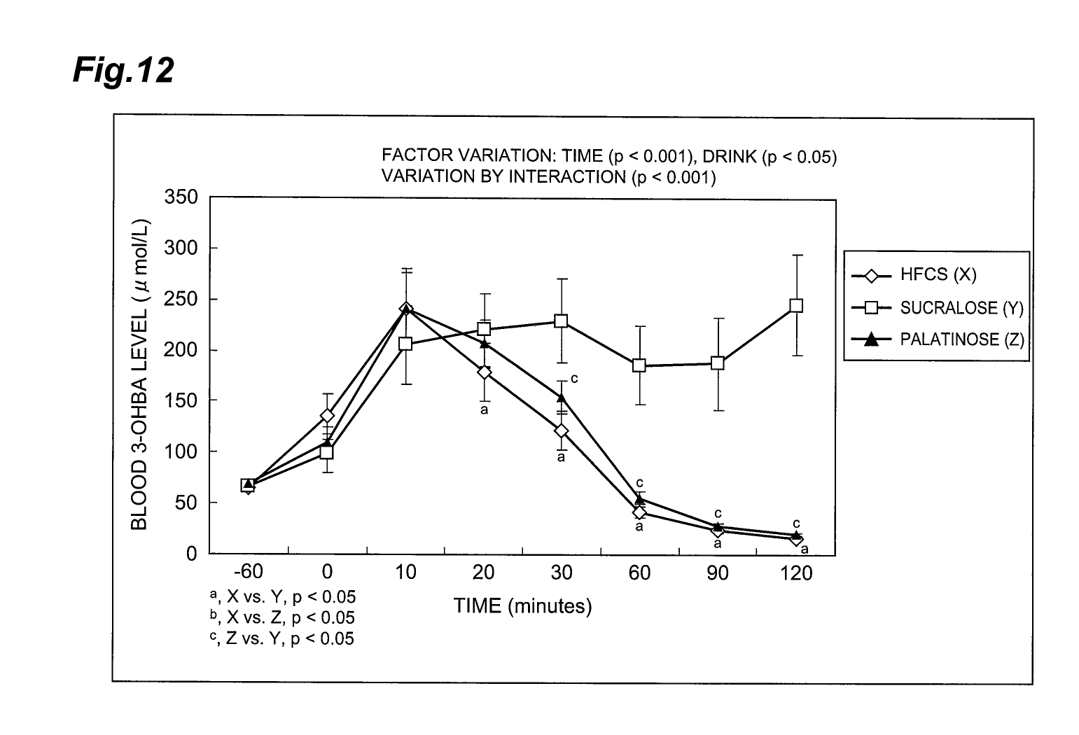

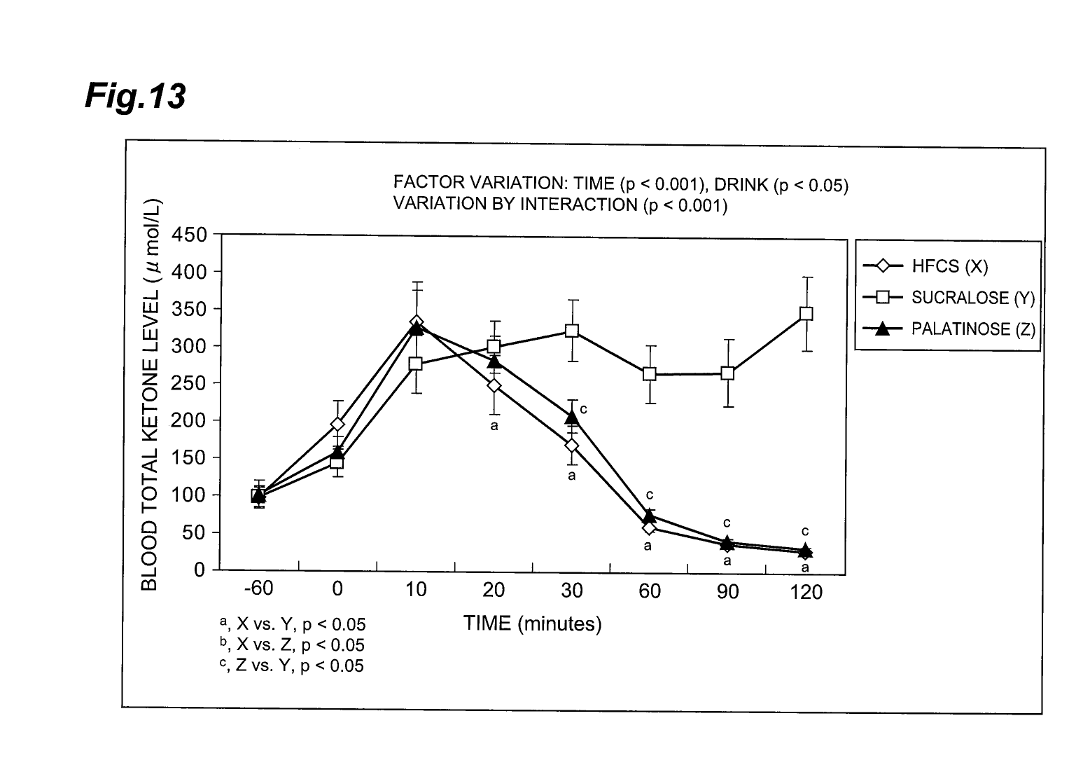

FIG. 12 and FIG. 13 show changes over time of blood 3-OHBA levels and blood total ketone bodies levels by drink intake after exercising. In taking sucralose, 3-OHBA and blood total ketone bodies rose together. This is because lipid oxidation is facilitated. On the other hand, in taking high-fructose corn syrup, 3-OHBA and blood total ketone bodies dropped. Surprisingly, it was found that in taking palatinose, ketone bodies dropped without their accumulation in blood although lipids ware oxidized (FIG. 8).

The above-mentioned facts revealed that in diabetic patients, patients with metabolic syndromes, and obese persons who are subject to a limited intake of carbohydrate, taking palatinose after exercising could maintain lipid metabolism and sufficient exercising effects as well as inhibit accumulation of ketone bodies. Therefore, it was shown that taking palatinose after exercising could prevent and improve ketosis or acidosis.

### Example 2

### Sugar Stick Containing Palatinose

Palatinose and sucrose having the same weight were mixed and filled in stick packs so that 3.5 g of palatinose and sucrose would be contained per pack, respectively.

### Example 3

### Gum Syrup Containing Palatinose

With the formulation shown in Table 2 below, gum syrup containing palatinose was produced. The production was conducted by putting palatinose and gum arabic together, conducting powder mixing, adding high-fructose corn syrup and water thereto, boiling and mixing. The solution above was adjusted to 30 by a Ref Brix scale.

TABLE 2

| Ingredient | g |
|---|---|
| Crystalline palatinose | 27.0 |
| (Crystalline Palatinose-IC, trade name, manufactured by | |
| Mitsui Sugar Co., Ltd.) | |
| high-fructose corn syrup | 48.0 |
| (EP-O, trade name, San-ei Sucrochemical Co., Ltd., 75%) | |
| Gum arabic | 4.0 |
| Water | 200.0 |

### Example 4

### Tablet Containing Palatinose

With the formulation shown in Table 3 below, a tablet containing palatinose was produced. The production was conducted by applying 300 kg/cm³ of tabletting pressure to the mixed powder with the formulation shown below to form a tablet with 18 mm of diameter, 5 mm of thickness and 1.5 g of weight.

TABLE 3

| Ingredient | Combination ratio (weight) |
|---|---|
| Powdered palatinose | 27.5 |
| (Paltinose Powder-ICP, trade name, | |
| manufactured by Mitsui Sugar Co., Ltd.) | |
| Powdered sugar | 27.5 |
| Citric acid | 1 |
| Sugar ester | 1 |
| Aspartame | 0.05 |
| Vitamin P | 0.0002 |
| Water | 0.6 |
| Lemon juice | Proper quantity |

### Example 5

### Powder Drink Containing Palatinose

With the formulation shown in Table 4 below, a powder drink containing palatinose was produced using a universal mixer according to a normal method. 50 g to 60 g of the obtained powder drink was dissolved in 250 ml to 1000 ml of water to prepare a refreshing drink.

TABLE 4

| | Combination ratio |
|---|---|
| Ingredient | (weight) |
| Crystalline palatinose | 42.35 |
| (Crystalline Palatinose-IC, trade name, | |
| manufactured by Mitsui Sugar Co., Ltd.) | |
| Sugar (granulated sugar) | 42.35 |
| Powder juice | 10 |
| Anhydrous citric acid | 3 |
| Sodium citrate | 0.4 |
| L-ascorbic acid | 0.5 |
| Sodium ascorbate | 0.3 |
| Riboflavin (content of 10% by weight) | 0.1 |

### Example 6

### Refreshing Drink (Sports Drink) Containing Palatinose

With the formulation shown in Table 5 below, a refreshing drink containing palatinose was produced. The production was conducted by dissolving the materials below in 250 ml of boiling water, followed by filling the resultant solution into a beverage can (for 250 ml).

TABLE 5

| Ingredient | g/can |
|---|---|
| Crystalline palatinose | 4 |
| (Crystalline Palatinose-IC, trade name, manufactured by | |
| Mitsui Sugar Co., Ltd.) | |
| Sugar (granulated sugar) | 4 |
| Citric acid | 0.15 |
| Vitamin C | 0.03 |
| Sodium chloride | 0.05 |
| Potassium chloride | 0.04 |
| Calcium chloride | 0.012 |
| Magnesium carbonate | 0.002 |
| Sodium glutamate | 0.006 |
| Stevia | 0.01 |
| Vitamin P | 0.0004 |
| Flavor | Proper quantity |

### Example 7

### Refreshing Drink (Sports Drink) Containing Palatinose

With the formulation shown in Table 6 below, a refreshing drink containing palatinose was produced. The production was conducted by dissolving the materials below in 250 ml of boiling water, followed by filling the resultant solution into a beverage can (for 250 ml).

TABLE 6

| | Combination ratio |
|---|---|
| Ingredient | (weight) |
| Crystalline palatinose | 10 |
| (Crystalline Palatinose-IC, trade name, | |
| manufactured by Mitsui Sugar Co., Ltd.) | |
| Citric acid | 0.15 |
| Vitamin C | 0.03 |
| Sodium chloride | 0.05 |
| Potassium chloride | 0.04 |
| Calcium lactate | 0.012 |
| Magnesium carbonate | 0.002 |
| Sodium glutamate | 0.006 |
| Flavor | Proper quantity |

### Example 8

### Refreshing Drink (Near Water) Containing Palatinose

With the formulation shown in Table 7 below, a refreshing drink containing palatinose was produced. The production was conducted by dissolving the materials below in 500 ml of boiling water, followed by filling the resultant solution into a plastic bottle (for 500 ml).

TABLE 7

| | Combination ratio |
|---|---|
| Ingredient | (weight) |
| Crystalline palatinose (Crystalline Palatinose-IC, trade | 5 |
| name, manufactured by Mitsui Sugar Co., Ltd.) | |
| Citric acid | 0.15 |
| Vitamin C | 0.03 |
| Sodium chloride | 0.05 |
| Potassium chloride | 0.04 |
| Calcium lactate | 0.012 |
| Magnesium carbonate | 0.002 |
| Sodium glutamate | 0.006 |
| Stevia | 0.005 |
| Vitamin P | 0.0002 |
| Flavor | Proper quantity |

### Example 9

### Powder Drink Containing Palatinose

With the formulation shown in Table 8 below, a powder drink containing palatinose was produced using a universal mixer according to a normal method. 50 g to 60 g of the obtained powder drink was dissolved in 250 ml to 1000 ml of water to prepare a refreshing drink.

TABLE 8

| | Combination ratio |
|---|---|
| Ingredient | (weight) |
| Crystalline palatinose (Crystalline Palatinose-IC, trade | 84.7 |
| name, manufactured by Mitsui Sugar Co., Ltd.) | |
| Powder juice | 10.0 |
| Anhydrous citric acid | 3.0 |
| Sodium citrate | 0.4 |
| L-ascorbic acid | 0.5 |
| Sodium ascorbate | 0.3 |
| Riboflavin (content of 10% by weight) | 0.1 |
| Flavor | Proper quantity |

### Example 10

### Jelly Drink Containing Palatinose

With the formulation shown in Table 9 below, a jelly drink containing palatinose (as a drink contained in a normal 180 g pouch) was produced.

TABLE 9

| Ingredient | Combination ratio (weight) |
|---|---|
| Crystalline palatinose (Crystalline | 15 |
| Palatinose-IC, trade name, manufactured | |
| by Mitsui Sugar Co., Ltd.) | |
| Dietary fiber | 5 |
| Gelling agent | 1 |
| Citric acid | 0.3 |
| Vitamin C | 0.06 |
| Sodium citrate | For pH adjustment |
| Flavor | Proper quantity |

The compounds shown in Examples 2 to 10 are ketone body accumulation inhibitors containing palatinose as an active ingredient.

1. A ketone body accumulation inhibitor containing isomaltulose as an active ingredient.

2. The ketone body accumulation inhibitor according to claim 1, for inhibiting accumulation of ketone bodies caused by exercising.
